(19) United States
(12) Patent Application Publication
(10) Pub. No.: US 2004/0082934 A1
(43) Pub. Date: Apr. 29, 2004

(54) Method of controlling pharmacokinetics of immunomodulatory compounds

(76) Inventor: Ronald J. Pettis, Cary, NC (US)

Correspondence Address: VENABLE LLP, 575 7TH STREET, N.W., WASHINGTON, DC, 20004-1601, US

(21) Appl. No.: 10/650,039

(22) Filed: Aug. 28, 2003

Related U.S. Application Data
(60) Provisional application No. 60/406,916, filed on Aug. 30, 2002.

(51) Int. Cl. A61M 31/00

(52) U.S. Cl. 604/506

(57) ABSTRACT

A method and device for administration of an immunomodulatory substance into skin.

### CROSS REFERENCE TO RELATED APPLICATION

This application claims priority to U.S. provisional application No. 60/406,916 filed Aug. 30, 2002, which is incorporated herein by reference.

### FIELD OF THE INVENTION

The invention constitutes a method and device for administration of an immunomodulatory compound into the intradermal space.

### BACKGROUND OF THE INVENTION

The importance of efficiently and safely administering pharmaceutical substances such as diagnostic agents and drugs has long been recognized. The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum, such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e. delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to at the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal space. Furthermore, even though the dermis, in particular the papillary dermis, has been known to have a high degree of vascularity, it has not heretofore been appreciated that one could take advantage of this high degree of vascularity to obtain an improved absorption profile for administered substances compared to subcutaneous administration. This is because small drug molecules are typically rapidly absorbed after administration into the subcutaneous tissue which has been far more easily and predictably targeted than the dermis has been. On the other hand, large molecules such as proteins are typically not well absorbed through the capillary epithelium regardless of the degree of vascularity so that one would not have expected to achieve a significant absorption advantage over subcutaneous administration by the more difficult to achieve intradermal administration even for large molecules.

One approach to administration beneath the surface to the skin and into the region of the intradermal space has been routinely used in the Mantoux tuberculin test. In this procedure, a purified protein derivative is injected at a shallow angle to the skin surface using a 27 or 30 gauge needle (Flynn et al, Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of substances.

Some groups have reported on systemic administration by what has been characterized as “intradermal” injection. In one such report, a comparison study of subcutaneous and what was described as “intradermal” injection was performed (Autret et al, Therapie 46:5-8, 1991). The pharmaceutical substance tested was calcitonin, a protein of a molecular weight of about 3600. Although it was stated that the drug was injected intradermally, the injections used a 4 mm needle pushed up to the base at an angle of 60°. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the substance would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al. administered sodium ceftazidime in what was characterized as “intradermal” injection using a 4 mm needle (Bressolle et al. J. Pharm. Sci. 82:1175-1178, 1993). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as an intradermal drug delivery device (U.S. Pat. No. 5,997,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into either into the dermis, the shallow SC or a combination thereof, nor did the reference suggest any possible pharmacokinetic advantage that might result from such selective administration.

To date, numerous therapeutic proteins and small molecular weight compounds have been delivered intradermally and used to effectively elicit a pharmacologically beneficial response. Most previous compounds (e.g. insulin, Neupogen, hGH, calcitonin) have been hormonal proteins. All administered proteins have exhibited several effects associated with ID administration, including more rapid onset of uptake and distribution (vs. SC) and in some case increased bioavailability. However, prior to the present invention, little or no information was known about the behavior of immunomodulatory compounds when administered intradermally.

### SUMMARY OF THE INVENTION

The present disclosure relates to a new parenteral administration method for immunomodulatory compounds based on directly targeting the dermal space whereby such method dramatically alters the pharmacokinetic (PK) and pharmacodynamic (PD) parameters of the administered compounds. The method includes administering the compounds to the dermal space either alone, or in conjunction with administration to the shallow subcutaneous space. The inventors have found that essentially simultaneous administration of an immunomodulatory compound to both the intradermal and subcutaneous space can produce especially efficacious results in comparison with the administration to either space by itself.

Immunomodulatory compounds that can be administered according to the invention include immunosuppressive agents, immunostimulatory agents and the like that have either a general or specific effect on the immune system of an individual. Effects of such compounds include a direct action on the immune system, or an indirect action which promotes an immunological response such as initiating an immunological cascade, or targeting a cell for destruction. J. Imunosuppressive agents are those that are generally administered to minimize unwanted immunological reactions (e.g. reduce autoimmunity, minimize transplant rejection, or suppress allergenic responses such as in allergy). Examples of such compounds include corticosteroids, such as prednisone; cytotoxic drugs, such as azathioprene or cyclophosphamide; other immunosuppressive agents such as cyclosporin A, FK506 (tacrolimus), and rapamycin; and monoclonal or polyclonal antibodies for immune rejection (horse anti-lymphocyte globulin), or autoimmune suppression (ex anti-TNF-α antibodies or binding proteins such as Enbrel®, or Remicade® infliximab). Immunostimulatory agents are those that are generally administered to enhance or promote desired innate or elicited immunological responses (e.g. chemotherapeutic agents, anti-infectives, vaccines, immune modulators). Examples of such compounds include tumor specific antibodies, vaccines of all types, interleukins, interferons. It should thus be appreciated that the invention is expected to be useful for administration of compounds that are considered to be immunoregulatory, pro-inflammatory, anti-inflammatory, chemoattractant, chemokinetic, cytokine, chemokine, chemotactic, haptotactic, and for agents that suppress these functions as well (e.g., suppression of chemokinesis, etc). Particularly preferred immunomodulatory substances include all classes of interferons (e.g. α, β, γ), all classes and subclasses of interleukins, anti-inflammatory agents, especially TNF α binding proteins, tumor targeting compounds, and bacterial cell wall components (such as lipopolysacharrides, BCG) and their synthetic derivatives.

By the use of direct intradermal (ID) administration means hereafter referred to as dermal-access means, for example, using microneedle-based injection and infusion systems (or other means to accurately target the intradermal space), the pharmacokinetics of many substances including drugs and diagnostic substances, and in particular immunomodulatory compounds, can be altered when compared to traditional parental administration routes of subcutaneous and intravenous delivery. These findings are pertinent not only to microdevice-based injection means, but other delivery methods such as needleless or needlefree ballistic injection of fluids or powders into the ID space, Mantoux-type ID injection, enhanced iontophoresis through microdevices, and direct deposition of fluid, solids, or other dosing forms into the skin if such delivery means can be accurately controlled to deposit the drug dose within the intradermal space. Disclosed is a method to increase the rate of uptake for parenterally-administered drugs without necessitating IV access. One significant beneficial effect of this delivery method is providing a shorter Tₘₐₓ (time to achieve maximum blood concentration of the drug). Potential corollary benefits include higher maximum concentrations for a given unit dose (Cₘₐₓ), higher bioavailability, more rapid uptake rates, more rapid onset of pharmacodynamics or biological effects, and reduced drug depot effects. According to the present invention, improved pharmacokinetics means increased bioavailability, decreased lag time (T_{lag}), decreased Tₘₐₓ, more rapid absorption rates, more rapid onset and/or increased Cₘₐₓ for a given amount of compound administered, compared to subcutaneous, intramuscular or other non-IV parenteral means of drug delivery. Decreases in T_{lag} and Tₘₐₓ, and more rapid absorption rates indicate faster onset for the therapeutic activity of drugs, while increased Cₘₐₓ and bioavailability indicate that more drug is present in systemic circulation, and generally indicate the potential for significant reduction of doses without loss of therapeutic effect.

By bioavailability is meant the total amount of a given dosage that reached the blood compartment. This is generally measured as the area under the curve in a plot of concentration vs. time. By “lag time” is meant the delay between the administration of a compound and time to measurable or detectable blood or plasma levels. Tₘₐₓ is a value representing the time to achieve maximal blood concentration of the compound, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a compound. However, numerical values can be determined more precisely by analysis using kinetic models (as described below) and/or other means known to those of skill in the art.

Directly targeting the dermal space, either alone or in combination with the shallow subcutaneous space, as taught by the invention, provides more rapid onset of effects of drugs and diagnostic substances. The inventors have found that substances can be rapidly absorbed and systemically distributed via controlled ID administration that selectively accesses the dermal vascular and lymphatic microcapillaries, thus the substances may exert their beneficial effects more rapidly than through SC administration alone. For example, the induction of high initial concentrations via ID injection coupled with induction of long duration circulating levels via shallow SC injection provides the ability to combine the two profiles to achieve both ends, potential targeting of a large immunomodulatory cell population via the dermal and lymphatic tissues, increased bioavailability, increased reproducibility.

Mammalian skin contains two layers, as discussed above, specifically, the epidermis and dermis. The epidermis is made up of five layers, the stratum corneum, the stratum lucidum, the stratum granulosum, the stratum spinosum and the stratum germinativum and the dermis is made up of two layers, the upper papillary dermis and the deeper reticular dermis. The thickness of the dermis and epidermis varies from individual to individual, and within an individual, at different locations on the body. For example, it has been reported that the epidermis varies in thickness from about 40 to about 90 μm and the dermis varies in thickness ranging from just below the epidermis to a depth of from less than 1 mm in some regions of the body to just under 2 to about 4 mm in other regions of the body depending upon the particular study report (Hwang et al., Ann Plastic Surg 46:327-331, 2001; Southwood, Plast. Reconstr. Surg 15:423-429, 1955; Rushmer et al., Science 154:343-348, 1966).

As used herein, intradermal is intended to mean administration of a substance into the dermis in such a manner that the substance readily reaches the richly vascularized papillary dermis and is rapidly absorbed into the blood capillaries and/or lymphatic vessels to become systemically bioavailable. Such can result from placement of the substance in the upper region of the dermis, i.e. the papillary dermis or in the upper portion of the relatively less vascular reticular dermis such that the substance readily diffuses into the papillary dermis. It is believed that placement of a substance predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of macromolecular and/or hydrophobic substances. Placement of the substance predominately at greater depths and/or into the lower portion of the reticular dermis is believed to result in the substance being slowly absorbed in the less vascular reticular dermis or in the subcutaneous region either of which would result in reduced absorption of macromolecular and/or hydrophobic substances. The controlled delivery of a substance in this dermal space below the papillary dermis in the reticular dermis, but sufficiently above the interface between the dermis and the subcutaneous tissue, should enable an efficient (outward) migration of the substance to the (undisturbed) vascular and lymphatic microcapillary bed (in the papillary dermis), where it can be absorbed into systemic circulation via these microcapillaries without being sequestered in transit by any other cutaneous tissue compartment. Additional potential benefits include directly targeting the immunomodulatory cells within the dermis and potentially lymphatic pathways, which may be involved in the uptake and distribution process.

By “shallow subcutaneous injection” is meant direct deposition of drugs into the shallow SC space. As mentioned above, persons of skill in the art will recognize that due to differences in individuals, and in the thickness of skin on various portions of the body within the same individual, shallow SC and intradermal depths are not fixed, and must be assessed for the circumstances of the injection(s). It is expected that persons of skill in the art will be able to make these determinations when necessary with no more than routine experimentation. Subject to the caveat above, “shallow subcutaneous injection” generally means that the injection is carried out at a depth of at least about 2 mm, more preferably, at least about 2.5 mm, up to a depth of no more than about 5 mm, more preferably, no more than about 4.0 mm.

Another benefit of the invention is to achieve more rapid systemic distribution and offset of administered substances. This is also pertinent for many hormones that in the body are secreted in a pulsatile fashion. Many side effects are associated with having continuous circulating levels of substances administered. A very pertinent example is female reproductive hormones that actually have the opposite effect (cause infertility) when continuously present in the blood.

Another benefit of the invention is to achieve higher bioavailabilities of administered substances. This effect has been most dramatic for ID administration of high molecular weight substances, especially proteins. The direct benefit is that ID administration with enhanced bioavailability, allows equivalent biological effects while using less active agent. This results in direct economic benefit to the drug manufacturer and perhaps consumer, especially for expensive protein therapeutics and diagnostics. Likewise, higher bioavailability may allow reduced overall dosing and decrease the patient's side effects associated with higher dosing.

Another benefit of the invention is the attainment of higher maximum concentrations of administered substances. The inventors have found that substances administered ID are absorbed more rapidly, with bolus administration resulting in higher initial concentrations. This is most beneficial for substances whose efficacy is related to maximal concentration. The more rapid onset allows higher C_{Max} values to be reached with lesser amounts of the substance. Therefore, the dose can be reduced, providing an economic benefit, as well as a physiological benefit since lesser amounts of the drug or diagnostic agent has to be cleared by the body.

Another benefit of the invention is no change in systemic elimination rates or intrinsic clearance mechanisms of administered substances. All studies to date by the applicants have maintained the same systemic elimination rate for the substances tested as via IV or SC dosing routes. This indicates this dosing route has no change in the biological mechanism for systemic clearance. This is an advantageous from a regulatory standpoint, since degradation and clearance pathways need not be reinvestigated prior to filing for FDA approval. This is also beneficial from a pharmacokinetics standpoint, since it allows predictability of dosing regimes. Some substances may be eliminated from the body more rapidly if their clearance mechanism are concentration dependent. Since ID delivery results in higher Cmax, clearance rate may be increased, although the intrinsic mechanism remains unchanged.

Another benefit of the invention is no change in pharmacodynamic mechanism or biological response mechanism. As stated above, administered drugs by the methods taught by the applicants still exert their effects by the same biological pathways that are intrinsic to other delivery means. Any pharmacodynamic changes are related only to the difference patterns of appearance, disappearance, and drug or diagnostic agent concentrations present in the biological system.

Using the methods of the present invention, pharmaceutical compounds and other substances, may be administered as a bolus, or by infusion. As used herein, the term “bolus” is intended to mean an amount that is delivered within a time period of less than ten (10) minutes. “Infusion” is intended to mean the delivery of a substance over a time period greater than ten (10) minutes. It is understood that bolus administration or delivery can be carried out with rate controlling means, for example a pump, or have no specific rate controlling means, for example user self-injection.

Another benefit of the invention is removal of the physical or kinetic barriers invoked when drugs passes through and becomes trapped in cutaneous tissue compartments prior to systemic absorption. Elimination of such barriers leads to an extremely broad applicability to various drug classes. Many drugs administered subcutaneously exert this depot effect—that is, the drug is slowly released from the SC space, in which it is trapped, as the rate determining step prior to systemic absorption, due to affinity for or slow diffusion through the fatty adipose tissue. This depot effect results in a lower Cₘₐₓ and longer Tₘₐₓ, compared to ID, and can result in high inter-individual variability of absorption. This effect is also pertinent for comparison to transdermal delivery methods including passive patch technology, with or without permeation enhances, iontophoretic technology, sonopheresis, or stratum corneum ablation or disruptive methods. Transdermal patch technology relies on drug partitioning through the highly impermeable stratum corneum and epidermal barriers. Few drugs except highly lipophilic compounds can breach this barrier, and those that do, often exhibit extended offset kinetics due to tissue saturation and entrappment of the drugs. Active transdermal means, while often faster than passive transfer means, are still restricted to compound classes that can be moved by charge repulsion or other electronic or electrostatic means, or carried passively through the transient pores caused by cavitation of the tissue during application of sound waves. The stratum corneum and epidermis still provide effective means for inhibiting this transport. Stratum corneum removal by thermal or laser ablation, abrasive means or otherwise, still lacks a driving force to facilitate penetration or uptake of drugs. Direct ID administration by mechanical means overcomes the kinetic barrier properties of skin, and is not limited by the pharmaceutical or physicochemical properties of the drug or its formulation excipients.

These and other benefits of the invention are achieved by directly targeting absorption by the papillary dermis and by controlled delivery of drugs, diagnostic agents, and other substances to the dermal space of skin. The inventors have found that by specifically targeting the intradermal space and controlling the rate and pattern of delivery, the pharmacokinetics exhibited by specific drugs can be unexpectedly improved, and can in many situations be varied with resulting clinical advantage.

In the case of a number of immunomodulatory compounds, such as α-interferon, the inventors have found that particularly advantageous results can be obtained by simultaneous intradermal administration of the compound at two different depths. Bolus administration of α-interferon at 1 and 3 mm depths resulted in a rapid early phase of blood levels followed by a sustained secondary phase that maintained α-interferon at levels in blood that were higher than those resulting from an equivalent dosage administered exclusively at 1 mm, 3 mm or subcutaneous depths.

By “simultaneous” administration is meant that delivery occurs such that uptake and maintenance of the delivered dosages will occur within the same time period and the pharmacokinetic profiles will be essentially superimposed. This does not necessarily require that delivery of the dosages occur at exactly the same time. In some cases they may be separated by time periods of seconds or minutes, but will generally always occur within 30 minutes of one another.

The present invention improves the clinical utility of ID delivery of drugs, diagnostic agents, and other substances to humans or animals. The methods employ dermalaccess means (for example a small gauge needle, especially microneedles), to directly target the intradermal space and to deliver substances to the intradermal space as a bolus or by infusion. It has been discovered that the placement of the dermal-access means within the dermis provides for efficacious delivery and pharmacokinetic control of active substances. The dermal-access means is so designed as to prevent leakage of the substance from the skin and improve adsorption within the intradermal space. The pharmacokinetics of immunomodulatory substances delivered according to the methods of the invention have been found to be vastly different to the pharmacokinetics of conventional SC delivery of the drug, indicating that ID administration according to the methods of the invention will provide improved clinical results. Delivery devices that place the dermal-access means at an appropriate depth in the intradermal space and control the volume and rate of fluid delivery provide accurate delivery of the substance to the desired location without leakage.

Disclosed is a method to increase the rate of uptake for parenterally-administered drugs without necessitating IV access. This effect provides a shorter Tₘₐₓ. Potential corollary benefits include higher maximum concentrations for a given unit dose (Cₘₐₓ), higher bioavailability, more rapid onset of pharmacodynamics or biological effects, and reduced drug depot effects.

It has also been found that by appropriate depth control of the dermal-access means within the intradermal space that the pharmacokinetics of immunomodulatory drugs delivered according to the methods of the invention can, if required, produce similar clinical results to that of conventional SC delivery of the drug.

The pharmacokinetic profile for individual compounds will vary according to the chemical properties of the compounds. For example, compounds that are relatively large, having a molecular weight of at least 1000 Daltons as well as larger compounds of at least 2000 Daltons, at least 4000 Daltons, at least 10,000 Daltons and larger and/or hydrophobic compounds are expected to show the most significant changes compared to traditional parenteral methods of administration, such as intramuscular, subcutaneous or subdermal injection. It is expected that small hydrophilic substances, on the whole, will exhibit similar kinetics for ID delivery compared to other methods.

In order to better control and refine the pharmacological results, delivery methods can be controlled by algorithms using logic components based on for example, physiologic models, rules based models or moving average methods, therapy pharmacokinetic models, monitoring signal processing algorithms, predictive control models, or combinations thereof. Such techniques are within the understanding of one of ordinary skill.

### BRIEF DESCRIPTION OF THE DRAWINGS

FIG. 1. Average profiles (n=3-6) for swine administered 3 MIU alpha-interferon (Schering) in 200 uL volume, either through single 1 or 3 mm microneedles, a dose split equally between 1 and 3 mm microneedles (100 uL each), or an equivalent dose delivered via standard SC method.

### DETAILED DESCRIPTION OF THE INVENTION

The present invention provides a method for therapeutic treatment by delivery of a drug or other substance to a human or animal subject by directly targeting the intradermal space, where the drug or substance is administered to the intradermal space through one or more dermal-access means incorporated within the device. Substances infused according to the methods of the invention have been found to exhibit pharmacokinetics superior to, and more clinically desirable than that observed for the same substance administered by SC injection.

The dermal-access means used for ID administration according to the invention is not critical as long as it penetrates the skin of a subject to the desired targeted depth within the intradermal space without passing through it. In most cases, the device will penetrate the skin to a depth of about 0.5-2 mm. However, in the case of certain types of compounds, the device may penetrate the skin to greater or more shallow depths for optimal results. The dermal-access means may comprise conventional injection needles, catheters or microneedles of all known types, employed singularly or in multiple needle arrays. The dermal-access means may comprise needleless devices including ballistic injection devices. The terms “needle” and “needles” as used herein are intended to encompass all such needle-like structures. The term microneedles as used herein are intended to encompass structures no larger than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. Dermal-access means also include ballistic fluid injection devices, powder-jet delivery devices, piezoelectric, electromotive, electromagnetic assisted delivery devices, gas-assisted delivery devices, of which directly penetrate the skin to provide access for delivery or directly deliver substances to the targeted location within the dermal space. By varying the targeted depth of delivery of substances by the dermal-access means, pharmacokinetic and pharmacodynamic (PK/PD) behavior of the drug or substance can be tailored to the desired clinical application most appropriate for a particular patient's condition. The targeted depth of delivery of substances by the dermal-access means may be controlled manually by the practitioner, or with or without the assistance of indicator means to indicate when the desired depth is reached. Preferably however, the device has structural means for controlling skin penetration to the desired depth within the intradermal space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

IV-like pharmacokinetics is accomplished by administering drugs into the dermal compartment in intimate contact with the capillary microvasculature and lymphatic microvasculature. In should be understood that the terms microcapillaries or capillary beds refer to either vascular or lymphatic drainage pathways within the dermal area.

While not intending to be bound by any theoretical mechanism of action, it is believed that the rapid absorption observed upon administration into the dermis is achieved as a result of the rich plexuses of blood and lymphatic vessels in the dermis. However, the presence of blood and lymphatic plexuses in the dermis would not by itself be expected to produce an enhanced absorption of macromolecules. This is because capillary endothelium is normally of low permeability or impermeable to macromolecules such as proteins, polysaccharides, nucleic acid polymers, substance having polymers attached such as pegylated proteins and the like. Such macromolecules have a molecular weight of at least 1000 Daltons or of a higher molecular weight of at least, 2000 Daltons, at least 4000 Daltons, at least 10,000 Daltons or even higher. Furthermore, a relatively slow lymphatic drainage from the interstitium into the vascular compartment would also not be expected to produce a rapid increase in plasma concentration upon placement of a pharmaceutical substance into the dermis.

One possible explanation for the unexpected enhanced absorption reported herein is that upon injection of substances so that they readily reach the papillary dermis an increase in blood flow and capillary permeability results. For example, it is known that a pinprick insertion to a depth of 3 mm produces an increase in blood flow and this has been postulated to be independent of pain stimulus and due to tissue release of histamine (Arildsson et al., Microvascular Res. 59:122-130, 2000). This is consistent with the observation that an acute inflammatory response elicited in response to skin injury produces a transient increase in blood flow and capillary permeability (see Physiology, Biochemistry, and Molecular Biology of the Skin, Second Edition, L. A. Goldsmith, Ed., Oxford Univ. Press, New York, 1991, p. 1060; Wilhem, Rev. Can. Biol. 30:153-172, 1971). At the same time, the injection into the intradermal layer would be expected to increase interstitial pressure. It is known that increasing interstitial pressure from values (beyond the “normal range”) of about −7 to about +2 mm Hg distends lymphatic vessels and increases lymph flow (Skobe et al., J. Investig. Dermatol. Symp. Proc. 5:14-19, 2000). Thus, the increased interstitial pressure elicited by injection into the intradermal layer is believed to elicit increased lymph flow and increased absorption of substances injected into the dermis.

By “improved pharmacokinetics” it is meant that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}). By enhanced absorption profile, it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration such as, for example, subcutaneous administration or intramuscular administration. In such comparisons, it is preferable, although not necessarily essential, that administration into the intradermal layer and administration into the reference site such as subcutaneous administration involve the same dose levels, i.e. the same amount and concentration of drug as well as the same carrier vehicle and the same rate of administration in terms of amount and volume per unit time. Thus, for example, administration of a given pharmaceutical substance into the dermis at a concentration such as 100 μg/ml and rate of 100 μL per minute over a period of 5 minutes would, preferably, be compared to administration of the same pharmaceutical substance into the subcutaneous space at the same concentration of 100 μg/ml and rate of 100 μL per minute over a period of minutes.

The enhanced absorption profile is believed to be particularly evident for substances that are not well absorbed when injected subcutaneously such as, for example, macromolecules and/or hydrophobic substances. Macromolecules are, in general, not well absorbed subcutaneously and this may be due, not only to their size relative to the capillary pore size, it may also be due to their slow diffusion through the interstitium because of their size. It is understood that macromolecules can possess discrete domains having a hydrophobic and/or hydrophilic nature. In contrast, small molecules which are hydrophilic are generally well absorbed when administered subcutaneously and it is possible that no enhanced absorption profile would be seen upon injection into the dermis compared to absorption following subcutaneous administration. Reference to hydrophobic substances herein is intended to mean low molecular weight substances, for example substances with molecular weights less than 1000 Daltons, which have a water solubility which is low to substantially insoluble

The above-mentioned PK and PD benefits are best realized by accurate direct targeting of the dermal capillary beds. This is accomplished, for example, by using microneedle systems of less than about 250 micron outer diameter, and less than 2 mm exposed length. Such systems can be constructed using known methods of various materials including steel, silicon, ceramic, and other metals, plastic, polymers, sugars, biological and or biodegradable materials, and/or combinations thereof.

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal space, the large exposed height of the needle outlet causes the delivered substance to be deposited at a much shallower depth nearer to the skin surface. As a result, the substance tends to effuse out of the skin due to backpressure exerted by the skin itself and to pressure built up from accumulating fluid from the injection or infusion. That is, at a greater depth a needle outlet with a greater exposed height will still seal efficiently where as an outlet with the same exposed height will not seal efficiently when placed in a shallower depth within the intradermal space. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of substances to the dermal space.

It has also been found that controlling the pressure of injection or infusion may avoid the high backpressure exerted during ID administration. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out of the skin. The appropriate delivery rates and volumes to obtain these effects for a selected substance may be determined experimentally using only ordinary skill. Increased spacing between multiple needles allows broader fluid distrubtion and increased rates of delivery or larger fluid volumes. In addition, it has been found that ID infusion or injection often produces higher initial plasma levels of drug than conventional SC administration, particularly for drugs that are susceptible to in vivo degradation or clearance or for compounds that have an affinity to the SC adipose tissue or for macromolecules that diffuse slowly through the SC matrix. This may, in many cases, allow for smaller doses of the substance to be administered via the ID route.

In another aspect, the present invention further provides for a method where the substance is delivered to a site which includes two or more compartments.

The present invention also provides for a method where the substance is delivered to multiple sites which each include one or more compartments.

The invention further provides for controlled delivery of a substance using algorithms having logic components which include physiologic models, rules based models or moving average methods, therapy pharmacokinetic models, monitoring signal processing algorithms, predictive control models, or combinations thereof.

In one embodiment, the present invention provides a method for combinations of shallow SC and ID delivery to achieve improved PK outcomes. These outcomes are not achievable using solely one delivery compartment or another. Individual or multiple site deposition via proper device configuration and/or dosing method may obtain unique and beneficial results. The utility of combining the effects of controlled shallow SC and ID delivery of substances using needles are previously unreported.

Devices for use with these methods can be configured to achieve both SC (or IM) and ID delivery.

The underlying technical principle is that the PK outcome of microneedle delivery is specific to the deposition depth and patterning of the administered fluid, that such deposition can be controlled mechanically via device design and engineering or by technique such as fluid overloading the ID space.

In addition, the invention includes needles (micro or otherwise) for SC injection having a length less than 5 mm length. Shallow SC delivery to a depth of about 3 mm yields almost identical PK to deep SC using traditional techniques. The utility of shallow SC delivery alone to yield more controlled profiles has never been exploited. In fact previously depths of less than 5 mm have been considered to not be within the SC space.

Mixed delivery either by device design or technique results in biphasic or mixed kinetic profiling. Minor differences in device length (1 mm vs. 2 mm vs. 3 mm) yield dramatic differences in PK outcomes. SC like profiles can be obtained with needle lengths often assumed to locate the end of the needle within the ID space. Shallow SC delivery is more consistent and uniform in PK outcomes than standard SC.

The administration methods useful for carrying out the invention include both bolus and infusion delivery of drugs and other substances to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver a substance the dermal-access means is placed adjacent to the skin of a subject providing directly targeted access within the intradermal space and the substance or substances are delivered or administered into the intradermal space where they can act locally or be absorbed by the bloodstream and be distributed systematically. The dermal-access means may be connected to a reservoir containing the substance or substances to be delivered. The form of the substance or substances to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. Delivery from the reservoir into the intradermal space may occur either passively, without application of the external pressure or other driving means to the substance or substances to be delivered, and/or actively, with the application of pressure or other driving means. Examples of preferred pressure generating means include pumps, syringes, elastomer membranes, gas pressure, piezoelectric, electromotive, electromagnetic pumping, or Belleville springs or washers or combinations thereof. If desired, the rate of delivery of the substance may be variably controlled by the pressure-generating means. As a result, the substance enters the intradermal space and is absorbed in an amount and at a rate sufficient to produce a clinically efficacious result.

As used herein, the term “clinically efficacious result” is meant a clinically useful biological response including both diagnostically and therapeutically useful responses, resulting from administration of a substance or substances. For example, diagnostic testing or prevention or treatment of a disease or condition is a clinically efficacious result. Such clinically efficacious results include diagnostic results such as the measurement of glomerular filtration pressure following injection of inulin, the diagnosis of adrenocortical function in children following injection of ACTH, the causing of the gallbladder to contract and evacuate bile upon injection of cholecystokinin and the like as well as therapeutic results, such as clinically adequate control of blood sugar levels upon injection of insulin, clinically adequate management of hormone deficiency following hormone injection such as parathyroid hormone or growth hormone, clinically adequate treatment of toxicity upon injection of an antitoxin and the like.

Having described the invention in general, the following specific but not limiting examples and reference to the accompanying Figure set forth various examples for practicing the dermal accessing, direct targeting drug administration method and examples of dermal administered drug substances providing improved PK and PD effects.

### EXAMPLE 1

Alpha-Interferon Delivery via Microneedles:

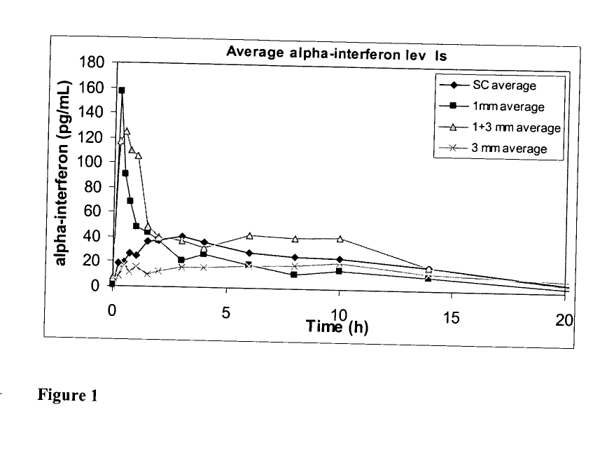

A feasibility trial with alpha-interferon was initiated to determine the effects of giving this compound via the ID route using microneedle devices, and also to demonstrate biphasic kinetics based on a specific mechanical device design. The drug was Schering Intron® A (interferon alfa-2b) at a concentration of 15 million international units (MIU)/mL, and was used as received in multi-unit dose cartridges. The administered dose in each condition was 200 uL of drug solution, for a total dose of 3 MIU/injection. Yucatan miniature swine (n=6) were injected in a crossover fashion, with each animal receiving doses via the IV and SC route via standard injection techniques. Microneedle injections were performed using single 34 G microneedles of either 1 mm or 3 mm length, or simultaneously through two independent microneedles, one each of length 1 and 3 mm, with half the total dose (100 uL) administered through each microneedle. Dosing rate was 50 uL/min from each microneedle for a total injection duration of 2 minutes for single microneedles, and 1 minute from the double microneedle systems. Plasma levels were assayed via a commercial immunoassay. The resulting detectable average plasma interferon levels are demonstrated in the graph below. Each curve represents the average of from n=3-6 animals. Data have been normalized to subtract background detection levels, but are not normalized for animal weight or total administered dose. Some curves were omitted due to incomplete of otherwise failed injections. The hypothesis that biphasic kinetics could be created specifically via device design is readily demonstrated in FIG. 1.

The 1 mm data show a classical “ID effect”: extremely rapid onset, high Cₘₐₓ, lower Tₘₐₓ, and a shortened systemic lifetime. The observed SC and 3 mm exhibit similar profiles with longer Tₘₐₓ, lower Cₘₐₓ, and a longer circulating lifetime. The average 3 mm data appear low in concentration but this is possible due to the limited # of replicates (n=3), and the fact that this was the final dose received by the animals over a multi-week study. The study swine could have been mounting an immunological antibody response to the administered human protein, which could affect detectable plasma levels. The combined microneedle delivery, splitting the dose between both the 1 and 3 mm microneedles, shows both the dramatic peak onset seen in the 1 mm alone case, and the longer circulating lifetime seen in the SC and 3 mm alone cases. This biphasic profile is effectively produced by addition of the two independent methods.

This system of administration should be effective for other immunomodulatory compounds such as other forms of interferon, as well as new chemical forms of interferon such as a pegylated version. The pegylated compound is expected to result in rapid onset but longer circulating half-life as a result of its chemical structure, which modulates systemic clearance. Devices that can administer to both tissue spaces may incorporate multiple needles of different lengths, single needles with multiple lumens or outlet ports, independent fluid paths, or flow controlled fluid paths such as those utilizing check valves to regulate flow between needles. Using the teachings of the present invention along with general knowledge in the art, skilled artisans will be able to design and make suitable devices with no more than routine experimentation.

The data presented herein reveal several novel aspects of previously uninvestigated areas:

1) Demonstration of the ID effect with an immunostimulant;

2) Demonstration of the ID effect with an interferon,

3) Demonstration of the ID effect with a compound representing the classes of immunostimulants, immunopotentiators, chemokines, cytokines, anti-viral agents, or other compound used for non-specific immuno-stimulation.

4) Demonstration of the ID effect for compounds with clinical indications for leukemia, melanoma, lymphoma, venereal or genital warts, AIDS related Kaposi's sarcoma, and chronic hepatitis B or chronic hepatitis C;

5) Kinetics of dermal delivery using an interferon;

6) Demonstration of the biphasic kinetics (early rapid onset with high peak level followed by longer lived sustained lower level) resulting from a preconceived device design (dual needle);

7) Demonstration of the microneedle delivery from a dual microneedle configuration targeting different tissue depths/different tissue types (shallow SC and ID)

Potential benefits of the invention include the following:

1) Therapeutic benefits related to rapidly achieving high concentration, and rapid onset;

2) Better dosing consistency both mechanically and pharmacokinetically;

3) Better control mechanisms for circadian or timed dosing control

4) A more patient-friendly dosing mechanism;

5) Potential benefits by directly targeting the immunomodulatory cells within the dermis and potentially lymphatic pathways, which may be involved in the uptake and distribution process;

6) Improved dosing for substances requiring both fast and long responses;

7) Potential enhanced bioavailability for alpha-interferon using the biphasic route or ID alone;

8) Simultaneous delivery of high loading dosage with a longer duration depot dose;

9) Rapid attainment of high circulating drug concentrations;

10) Reduced dosage (drug amount) for the patient;

11) Reduced manufacturing capacity needed to obtain an equivalent number of doses; and

12) More predictable dosing across the patient population.

The results show that the relative bioavailability of α interferon is increased when administered simulataneously at intradermal (1 mm) and shallow subcutaneous (3 mm) depths. The resulting dose-sparing effect will allow administration of dosages that are lower than has been possible with the standard subcutaneous injection method of the art, resulting in a large cost saving to pharmaceutical manufacturers and consumers.

In general, ID and shallow SC delivery as taught by the methods described herein via dermal access microneedle devices provides a readily accessible and reproducible parenteral delivery route, with high bioavailability, as well as the ability to modulate plasma profiles by adjusting the device infusion parameters, since uptake is not rate-limited by biological uptake parameters.

In the previously described examples, the methods practiced by the invention demonstrate the ability to deliver an immunomodulatory substance in vivo with greatly improved clinical efficacy. This data indicates an improved pharmacological result for ID administration of these substances, either alone or together with shallow SC injection, would be expected.

All references cited in this specification are hereby incorporated by reference. The discussion of the references herein is intended merely to summarize the assertions made by their authors and no admission is made that any reference constitutes prior art relevant to patentability. Applicants reserve the right to challenge the accuracy and pertinency of the cited references.

What is claimed is:

1. A method for directly delivering an immunomodulatory substance into an intradermal space within mammalian skin comprising administering the substance through at least one hollow needle having an outlet with an exposed height between 0 and 1 mm, said outlet being inserted into the skin to a depth of between 0.3 mm and 2 mm, such that delivery of the substance occurs at a depth between 0.3 mm and 2 mm.

2. The method according to claim 1 wherein the delivered substance has improved pharmacokinetics compared to pharmacokinetics after subcutaneous injection.

3. The method of claim 1 wherein the administration is through at least one small gauge hollow needle.

4. The method of claim 1 wherein the needle has an outlet with an exposed height between 0 and 1 mm.

5. The method of claim 1 wherein injecting comprises inserting the needle to a depth which delivers the substance at least about 0.3 mm below the surface to no more than about 2 mm below the surface.

6. The method of claim 1 wherein administering comprises inserting the needle into the skin to a depth of at least about 0.3 mm and no more than about 2 mm.

7. The method of claim 2 wherein the improved pharmacokinetics is increased bioavailability of the substance.

8. The method of claim 2 wherein the improved pharmacokinetics is a decrease in Tₘₐₓ.

9. The method of claim 2 wherein the improved pharmacokinetics is an increase in Cₘₐₓ.

10. The method of claim 2 wherein the improved pharmacokinetics is a decrease in T_{lag}.

11. The method of claim 2 wherein the improved pharmacokinetics is enhanced absorption rate.

12. The method of claim 1 wherein the substance is administered over a time period of not more than ten minutes.

13. The method of claim 1 wherein the substance is administered over a time period of greater than ten minutes.

14. The method of claim 1 wherein the substance is a protein or peptide.

15. The method of claim 1 wherein the substance is administered at a rate between 1 nL/min. and 200 mL/min.

16. The method of claim 1 wherein said substance is an immunostimulant.

17. The method of claim 1 wherein said substance is an immunosuppressant.

18. The method of claim 1 wherein said substance is selected from the group consisting of an interferon, an interleukin, an anti-inflammatory agent, a tumor targeting compound, and a bacterial cell wall component or synthetic derivative thereof.

19. The method of claim 18 wherein said substance is a lipopolysacharride or BCG, or a synthetic derivative thereof.

20. The method of claim 18 wherein said substance is α interferon.

21. The method of claim 1 wherein the needle(s) are inserted substantially perpendicularly to the skin.

22. A method of administering an immunomodulatory substance comprising injecting or infusing the substance intradermally through one or more microneedles having a length and outlet suitable for selectively delivering the substance into the dermis to obtain absorption of the substance in the dermis.

23. The method of claim 22 wherein absorption of the substance in the dermis produces improved systemic pharmacokinetics compared to subcutaneous administration.

24. The method of claim 23 wherein the improved pharmacokinetics is increased bioavalability.

25. The method of claim 23 wherein the improved pharmacokinetics is decreased Tₘₐₓ.

26. The method of claim 23 wherein the improved pharmacokinetics is an increase in Cₘₐₓ.

27. The method of claim 23 wherein the improved pharmacokinetics is a decrease in T_{lag}.

28. The method of claim 23 wherein the improved pharmacokinetics is an enhanced absorption rate.

29. The method of claim 22 wherein the length of the microneedle is from about 0.5 mm to about 1.7 mm.

30. The method of claim 22 wherein the microneedle is 30 gauge or narrower.

31. The method of claim 22 wherein the microneedle has an outlet of from 0 to 1 mm.

32. The method of claim 22 wherein the microneedle is configured in a delivery device which positions the microneedle perpendicular to skin surface.

33. The method of claim 22 wherein the microneedle needle is contained in an array of microneedles needles.

34. The method of claim 33 wherein the array comprises 3 microneedles.

35. The method of claim 33 wherein the array comprises 6 microneedles.

36. The method of claim 22 wherein said substance is an immunostimulant.

37. The method of claim 22 wherein said substance is an immunosuppressant.

38. The method of claim 22 wherein said substance is selected from the group consisting of an interferon, an interleukin, an anti-inflammatory agent, a tumor targeting compound, and a bacterial cell wall component or synthetic derivative thereof.

38. The method of claim 38 wherein said substance is a lipopolysacharride or BCG, or a synthetic derivative thereof.

40. The method of claim 38 wherein said substance is α interferon.

41. A method for delivering an immunomodulatory substance to a subject comprising: contacting the skin of the subject with a device having a dermal-access means for accurately targeting the dermal space of the subject with an efficacious amount of the bioactive substance.

42. The method of claim 41 wherein the pharmacokinetics of the immunomodulatory substance is improved relative to the pharmacokinetics of the substance when administered subcutaneously.

43. The method of claim 42 wherein the improved pharmacokinetics is an increase in bioavailability.

44. The method of claim 42 wherein the improved pharmacokinetics is a decrease in Tₘₐₓ.

45. The method of claim 42 wherein the improved pharmacokinetics comprises an increase in Cₘₐₓ of the substance compared to subcutaneous injection.

46. The method of claim 42 wherein the improved pharmacokinetics is a decrease in T_{lag}.

47. The method of claim 42 wherein the improved pharmacokinetics is an enhanced absorption rate.

48. The method of claim 41 wherein the dermal access means comprises one or more hollow microcannula having a length of from about 0.3 to about 2 mm.

49. The method of claim 41 wherein said dermal access means comprises one or more hollow microcannula having an outlet with an exposed height between 0 and 1 mm.

50. The method of claim 41 wherein the substance is an immunostimulant.

51. The method of claim 41 wherein said substance is an immunosuppressant.

52. The method of claim 41 wherein said substance is selected from the group consisting of an interferon, an interleukin, an anti-inflammatory agent, a tumor targeting compound, and a bacterial cell wall component or synthetic derivative thereof.

53. The method of claim 52 wherein said substance is a lipopolysacharride or BCG, or a synthetic derivative thereof.

54. The method of claim 52 wherein the substance is a interferon.

55. A method for delivering an immunomodulatory substance into tissue comprising delivering the substance within or beneath the skin at least into the intradermal space to access one or more compartments, which compartments afford the substance different pharmacokinetics, which enhance the effectiveness of the substance in terms of a resultant composite pharmacokinetics.

56. The method of claim 55 wherein the substance is delivered to a site which includes two or more of the compartments.

57. The method of claim 55 wherein the substance is delivered to multiple sites which each include one or more of the compartments.

58. The method of claim 55 wherein the delivery of the substance is by a needle or cannula.

59. The method of claim 58 wherein a single needle is inserted into the intradermal space.

60. The method of claim 58 wherein multiple needles or needle arrays are inserted into the intradermal space.

61. The method of claim 60 wherein the multiple needles have different lengths.

62. The method of claim 58 wherein the needle is about 300 μm to about 5 mm long.

63. The method of claim 62 wherein the needle is about 500 μm to about 1 mm long.

64. The method of claim 59 wherein the needle has an outlet placed at a depth of about 300 μm to about 2 mm when the needle is inserted into the intradermal space.

65. The method of claim 64 wherein the outlet is at a depth of about 500 μm to about 1.7 mm when the needle is inserted.

65. The method of claim 65 wherein the outlet is at a depth of about 750 μm to about 1.5 mm when the needle is inserted.

66. The method of claim 64 wherein the outlet has an exposed height of about 0 to about 1 mm.

67. The method of claim 66 wherein the outlet has an exposed height of about 0 to about 300 μm.

68. The method of claim 55 wherein the delivery of the substance is by a needle selected from the group consisting of microneedles, catheter needles, and injection needles.

69. The method of claim 55 wherein said substance is an immunostimulant.

70. The method of claim 55 wherein said substance is an immunosuppressant.

71. The method of claim 55 wherein said substance is selected from the group consisting of an interferon, an interleukin, an anti-inflammatory agent, a tumor targeting compound, and a bacterial cell wall component or synthetic derivative thereof.

72. The method of claim 71 wherein said substance is a lipopolysacharride or BCG, or a synthetic derivative thereof.

73. The method of claim 71 wherein the substance is α interferon.

74. The method of claim 55 wherein the substance is infused.

75. The method of claim 55 wherein the substance is delivered as a bolus.

76. The method of claim 55 further comprising controlling the delivery rate or volume delivered of the substance to the intradermal space.

77. The method of claim 55 wherein said delivery is controlled pursuant to an algorithm having logic components which include physiologic models, rules based models or moving average methods, therapy pharmacokinetic models, monitoring signal processing algorithms, predictive control models, or combinations thereof.

78. The method of claim 55 wherein the substance is infused at a constant rate.

79. The method of claim 55 wherein the substance is delivered by a combination of infusion and bolus injection.

80. The method of claim 55 wherein the multiple compartments are intradermal and subcutaneous tissue compartments.

81. The method of claim 55 wherein the access of the one or more compartments is by at least one needle that targets the ID compartment and at least another one needle that targets SC compartment.

82. The method of claim 55 wherein the delivery to the one or more compartments is by at least one needle which essentially simultaneously targets the ID compartment and SC compartment.

83. The method of claim 55 wherein the delivery of the one or more compartments is by at least one needle which sequentially targets the ID compartment and SC compartment.

84. The method of claim 55 wherein the delivery to multiple compartments is by at least one needle that targets the interface of the ID and SC compartments.

85. The method of claim 55 wherein a portion of said therapeutic substance is absorbed more rapidly into the intradermal tissue space, and the remaining portion is absorbed less rapidly into the subcutaneous tissue space.

86. The method of claim 55 wherein the enhanced effectiveness is a decrease in Tₘₐₓ.

87. The method of claim 55 wherein the enhanced effectiveness is an increase in Cₘₐₓ.

88. The method of claim 55 wherein the enhanced effectiveness is a decrease in T_{lag}.

89. The method of claim 55 wherein the enhanced effectiveness is enhanced absorption rate.

90. The method of claim 55 wherein the enhanced effectiveness is improved bioavailability.
